(19) United States
(12) Patent Application Publication
(10) Pub. No.: US 2026/0144611 A1
(43) Pub. Date: May 28, 2026

(54) Skin Marking Device

(71) Applicant: Santiago Krumm Cabezas, Needham, MA (US)

(72) Inventor: Santiago Krumm Cabezas, Needham, MA (US)

(21) Appl. No.: 19/453,286

(22) Filed: Jan. 20, 2026

Related U.S. Application Data
(63) Continuation-in-part of application No. 18/412,763, filed on Jan. 15, 2024, now Pat. No. 12,544,102.

(51) Int. Cl. A61B 90/00; A61B 8/00; A61B 17/34

(52) U.S. Cl. CPC A61B 90/39; A61B 17/3403; A61B 8/4209; A61B 2017/3413; A61B 2090/3908

(57) ABSTRACT

A skin marking device is provided. The skin marking device has an outer frame that can be fitted over an ultrasound probe. The ultrasound probe can be used to identify a location for a needle or other medical device. Once the location is identified, the probe may be removed while keeping the frame in place. A skin-marking tool may then be fitted with/into the frame to mark the skin, thereby identifying the precise location for action.

### BACKGROUND OF THE INVENTION

The present disclosure relates generally to skin marking devices. More particularly, the present disclosure relates to a device which may use a frame to identify a location for needle placement based on ultrasound, and a marking device which fits within the frame to mark the skin for subsequent needle placement.

Placement of spinal, epidural, or other needles, in the epidural or intrathecal space can sometimes be challenging due to bony structures obstructing the path of the needle; finding the correct insertion site that will allow a direct, unobstructed path for the needle can sometimes be difficult.

In the prior art, technicians sometimes use their fingers to feel the spacing between bones. This can be difficult and unreliable in many cases due to surrounding muscle, fat, and inherent imprecision of doing things by “feel.” Other practices include using an ultrasound probe, however, in many instances, the ultrasound probe must be removed from the skin after identifying an adequate space for placement, and then needle placement occurs after removal of the ultrasound probe. US imaging of the spine allows for identification of gaps within the bony spine, but the technician must then mark the area with a marker, pen, or the like, and then remove the probe. The optimal area is inherently at the center point of the ultrasound probe which cannot be directly accessed without removing the probe. Ultrasound jelly makes marking difficult, because one cannot mark well through it, and marks can be erased when gel is wiped off the skin; more importantly, the ultrasound probe must be removed from the skin, to access the point on the skin that corresponds to the center point of the probe when the appropriate image for a space for needle insertion is seen on screen, and the technician can only estimate the spot where the center of the probe was before it was removed.

Therefore, what is needed is a system which can mark a location for needle insertion reliably after the location is identified by an ultrasound or other imaging device.

### SUMMARY OF THE INVENTION

The subject matter of this application may involve, in some cases, interrelated products, alternative solutions to a particular problem, and/or a plurality of different uses of a single system or article.

In one aspect, a skin-marking assembly is provided. The skin marking assembly has an outer frame sized to fit closely around an outer perimeter of an ultrasound probe. The assembly further having a marking tool which has top, bottom, left and right edges. These top, bottom, left edge, and right edge are each sized to slidably fit within an inner perimeter of the outer frame. Though in other similar aspects the marking tool may fit to any part of the frame without straying from the scope of this disclosure. The marking tool may further have a protrusion on a front side. The protrusion is sized and configured to form an indent in the skin of a patient when pushed into the skin, so as to mark the skin.

In another aspect, the skin marking assembly has an outer frame sized to fit closely around an outer perimeter of an ultrasound probe. The assembly, further having a marking tool which has top, bottom, left and right edges. These top, bottom, left edge, and right edge are each sized to slidably fit within an inner perimeter of the outer frame. Though in other similar aspects the marking tool may fit to any part of the frame without straying from the scope of this disclosure. The marking tool may further have a protrusion on a front side. This protrusion defines an open shape such that a central opening is defined at a front of the protrusion.

In yet another aspect, a method of marking an area of a patient for placement of an epidural or the like is provided. The method begins with placing or otherwise connecting a frame of a skin-marking assembly on an ultrasound probe. The ultrasound probe may then be used with an ultrasound device to identify a desired location for needle placement, such as a spacing between vertebrae of a patient. Once identified, the frame can be held in place in the position identified and the ultrasound probe can be removed from the frame and away from the patient. A marking tool may then be placed into the frame or connected to the frame. Then, this marking tool can be used for marking the skin of the patient at a location corresponding to the center point of the ultrasound probe.

### BRIEF DESCRIPTION OF DRAWINGS

FIG. 1 provides a perspective view of an embodiment of the present disclosure.

FIG. 2 provides a perspective view of another embodiment of the present disclosure.

FIG. 3 provides a perspective view of still another embodiment of the present disclosure in use.

FIG. 4 provides a perspective view of yet still another embodiment of the present disclosure in use.

FIG. 5 provides a view of an embodiment of the present disclosure.

FIG. 6 provides a view of an embodiment of use of yet another embodiment of the present disclosure.

FIG. 7 provides a perspective view of another embodiment of the present disclosure.

FIG. 8 provides a perspective view of yet another embodiment of the present disclosure.

FIG. 9 provides a perspective view of yet another embodiment of the present disclosure in use.

FIG. 10 provides another perspective view of yet another embodiment of the present disclosure in use.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns a skin marking device, also referred to as a skin marking assembly. The present disclosure relates to a device which allows precise marking on a patient as to where to place a needle, cannula, other medical tools, and the like. This system greatly limits the chance of improper needle (and other) placement, creating better and safer patient outcomes.

The system involves, in most embodiments, two parts-a frame which fits around, or partially around, or otherwise engages with a probe of an ultrasound, and a marking tool which engages with the frame and can be used to mark, or guide a marker on the skin of the patient. The mark made using the marking tool can then be used to guide placement of a needle and the like. These components may vary in size, shape, and configuration depending on manufacturer and type of ultrasound probe used.

In many embodiments, the frame is formed of a rectangular, oval, or other similar open shape which has an inside perimeter which fits around the outside perimeter of the ultrasound probe. Other embodiments may use a non-closed shape, like an open frame which connects to only one, two, or three sides of the probe.

In many embodiments, the skin marking tool is sized to precisely engage with the frame. For example, the marking tool may have an outer perimeter, or extending arms, which fit to the frame in only one orientation such that the skin marking can only be made in one place based on frame position. This eliminates the risk of human error in the marking process. For example, the marking tool may have a shape that only allows for it to fit to the frame in one orientation. Preferably, the tool will have at least two points of contact with the frame to ensure that correct orientation of the marking is achieved.

In one embodiment of operation, a method of using the device disclosed herein for marking an area of a patient for placement of an epidural needle (among other operational uses) may first involve placing a frame of a skin-marking assembly over an ultrasound probe. In this embodiment, the ultrasound probe is placed on the skin of the patient with the outer frame of the skin marking assembly fit around the probe. The probe is then manipulated to identify a desirable location for the placement of the epidural, with the frame moving with the ultrasound probe. The location may be identified as, e.g. a spacing between spinous processes of the vertebrae of the patient. The technician may then advance the frame over the probe that is in contact with the skin at the desired location, so that the frame makes contact with the skin, and while holding the frame in place, pressure can be made with the frame against the skin. In some embodiments, when the frame is pressed against the patient, outer indentations can be made on the skin. After this, the ultrasound probe is removed. Next, while holding the frame in place, the marking tool can then be placed into the frame to make a central mark on the patient's skin in the desired area as identified by the ultrasound probe. Typically, the marking tool is configured to mark the skin at a location where the center of the ultrasound probe was. The marking tool may then be used to mark the skin and/or as a needle guide. Marking may be by pushing a protrusion on the tool into the skin, using a marker or other marking device (which may be either mounted to the marking tool or used in conjunction with the marking tool). In marking embodiments, the entire assembly may be removed because the mark has been made on the patient for subsequent epidural placement. In other embodiments wherein the marking tool is used as a needle guide, the assembly may remain in place, or the frame may be removed.

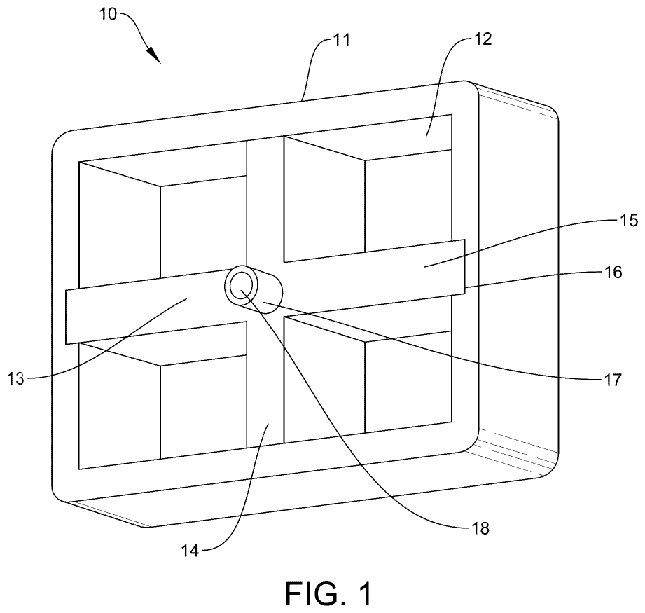

Turning now to FIG. 1, a perspective view of an embodiment of the skin marking assembly is provided. The skin marking assembly 10 has an outer frame 11 which fits around an ultrasound probe, and a marking tool 13 which fits within the frame 11. As shown below, the frame 11 fits around the ultrasound probe for proper positioning. The probe can be removed and the skin marking tool 13 fits within the inner perimeter 12 of the frame 11. The marking tool 13, in this embodiment, has a cross shape. Of course, any other shape may be used without straying from the scope of this invention.

For example, in this embodiment, the marking tool 13 has vertical arms 14 and horizontal arms 15 where both the vertical arms 14 and horizontal arms 15 contact the inner perimeter 12 of the frame 11. However, in other embodiments, the vertical arms 14 may be shortened so as not to contact the inner perimeter 12 or be eliminated entirely, leaving a marking tool 13 with only horizontal arms 15. Still referring to FIG. 1, the horizontal arms 15, in this embodiment, fit into slots 16 defined by the inner perimeter 12 of the outer frame 11. Of course, other configurations such as slots to engage with the top and bottom vertical arms 14 in addition or instead may also be used. In this view, the vertical arms 14 frictionally fit with the inner perimeter 12 wall of the frame 11 or have a small gap therebetween. The slots 16 ensure proper positioning and provide a secure path for marking.

In this embodiment, the slots 16 and the horizontal arms 15 are rectangular in shape, but in other embodiments, either the slots 16, the horizontal arms 15, or both may be varied in size and shape to allow for easier insertion and marking. For example, in one alternative embodiment, at least one of the slots 16 is Y-shaped, with a wider opening on the side of the frame 11 where the marking tool 13 is inserted, tapering to a narrow rectangular channel and opening on the side of the frame 11 intended for marking. This exemplary alternative configuration may allow for easier insertion of the marking tool 13 through a wider opening, and the tapered Y-shape of the slot(s) 16 may more effectively guide the horizontal arms 15 through a straight channel to the final marking spot in the center of the frame 11. Of course, this alternative configuration may be used either in combination with or exclusion of other embodiments disclosed herein, including, but not limited to, vertical arms 14 and slots on the top and bottom of the inner perimeter 12.

Referring back to FIG. 1, the marking tool 13 further has a protrusion 17 which extends from front and rear (not shown) of the marking tool 13. The protrusion 17 can be used to press into the skin, making an indent or mark. In the embodiment shown, the protrusion 17 defines a central opening 18 which has been found to provide a better marking by providing increased pressure on the non-open space of the protrusion 17. In further embodiments, this central opening 18 may extend from front to back all the way through the marking tool 13. In such an embodiment, a needle may be passed through the central opening 18 using the marking tool 13 as a needle guide. In other embodiments (not shown), the protrusion 17 may only extend from either the front or the rear of the marking tool 13, not both.

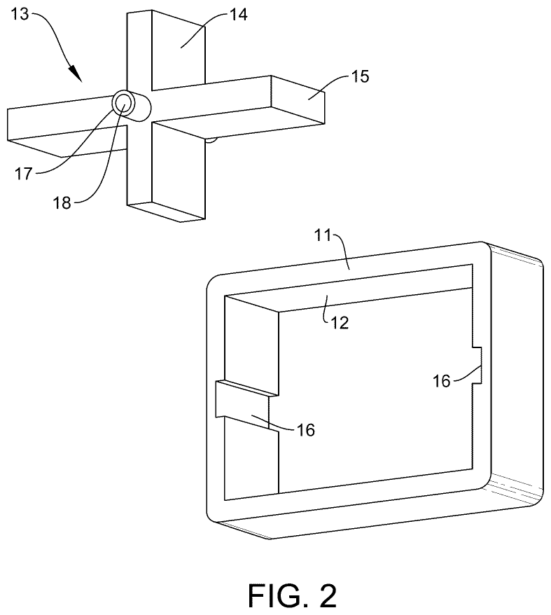

FIG. 2 shows another embodiment of the present disclosure having the frame and marking tool separated. The frame 11 has an inner perimeter 12 which defines slots 16 on opposite vertical walls, facing each other and vertically aligned with each other. As noted, the slots may take other orientations such as being off set, being Y-shaped, triangular or straight, being formed as channels to hold the tool in place (such as a T-shaped channel), being on the horizontal walls, and the like, without straying from the scope of this invention. The marking tool 13 is shown in this embodiment as a cross-shaped device with vertical arms 14 which are sized to fit very closely to the top and bottom inner perimeter 12 of the frame 11 to ensure a proper vertical positioning of the protrusion 17 which is used to mark the skin of the patient. Similarly, the horizontal arms 15 of the marking tool 13 are sized to fit into the slots 16 formed by the frame 11 which ensures a proper horizontal (and vertical) positioning of the protrusion 17. As can be somewhat seen in this embodiment, the tool 13 also has a protrusion 18 on its rear side as well. The two-sided protrusion configuration may make tool use easier as it can be used in the same way in either direction, limiting time and effort needed to properly orient. In some embodiments, the vertical arms 14 are sized to have a spacing or gap between their ends and the inner surface of the frame 11, while the horizontal arms 15 may fit into the slots 16. Such a configuration may make it easier to fit the marking tool 13 into the frame 11. In other embodiments, the vertical arms 14 may fit tightly to the inner surface of the frame 11.

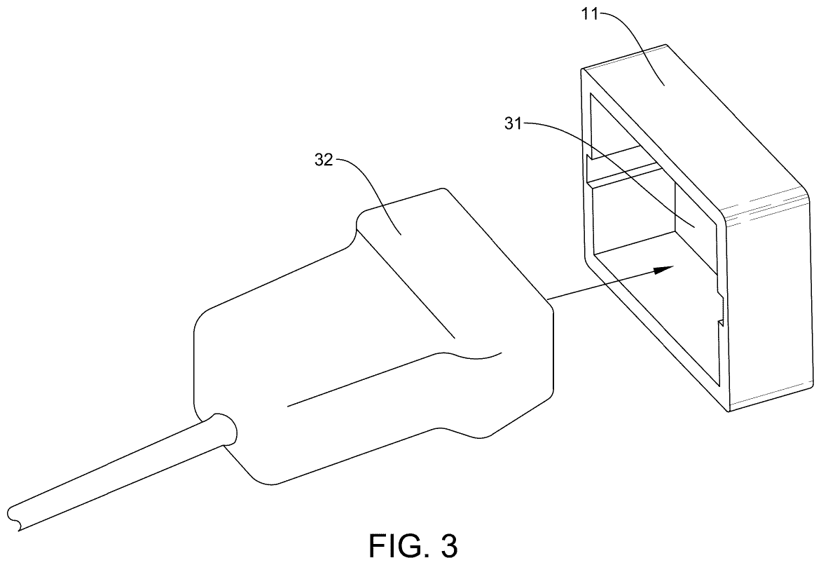

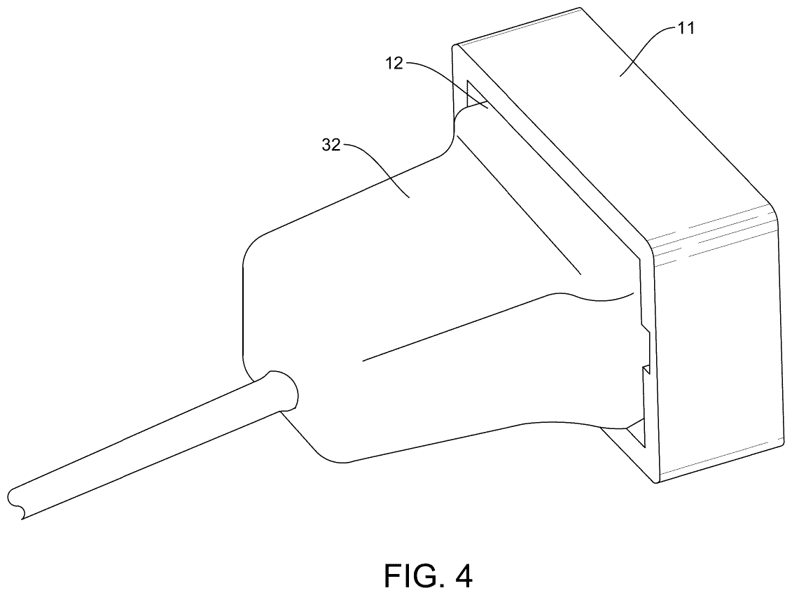

FIGS. 3 and 4 provide views of use of the ultrasound probe and frame together. The frame 11 is selected to fit closely around the front of the ultrasound probe 31 and defines an inner space 31 into which the probe may fit. By closely fitting around the probe 32, the frame 11 will move with the probe as the desired location is found, and will provide a precise alignment for later placement of the marking tool. If excessive gaps are present between the frame 11 and probe 32, the frame 11 could be moved out of proper alignment, leading to an incorrect marking location and in turn incorrect needle placement. As noted, once the desired location is found, the probe 32 is removed, and the frame 11 held in place for subsequent placement of the marking tool.

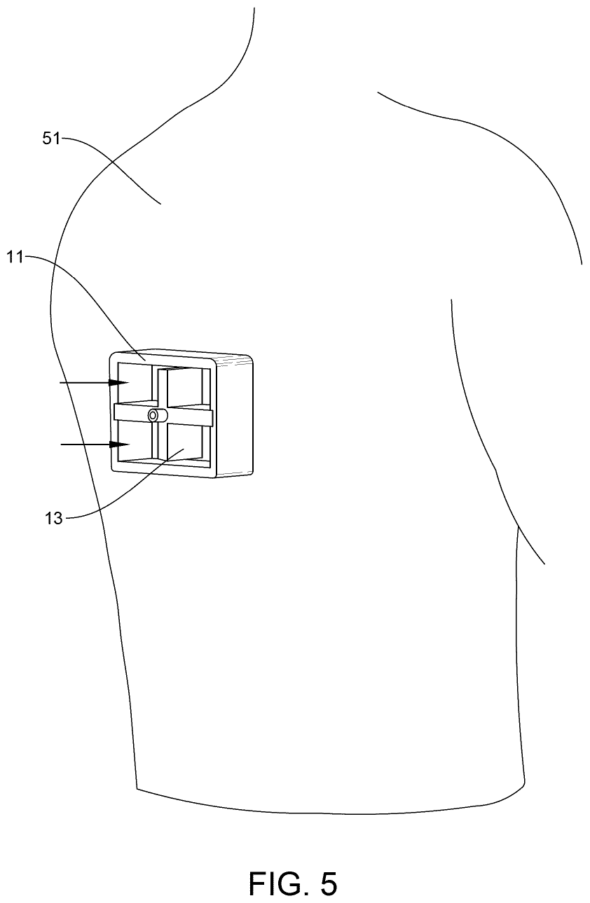

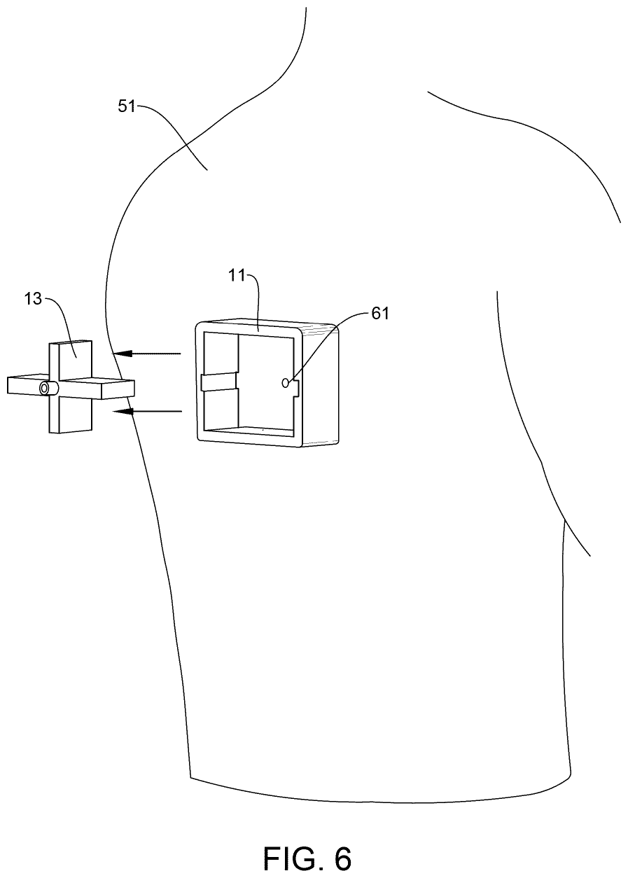

FIGS. 5 and 6 show an embodiment of the skin marking device in use after the ultrasound probe has been removed. The marking tool 13 is placed into the frame 11 which is held in place on the patient 51. The tool, in this embodiment, is pressed into the skin of the patient in the direction of arrows. Of course, other marking systems may be used such as ink, a spray, and the like. The protrusion of the tool will make a mark or indent 61 in the skin of patient 51, which corresponds to the center of the screen when the ultrasound probe obtained the desired image and aligned the target with the center of the ultrasound probe. After the indent 61 is made, this in turn allows removal of the device (frame 11 and tool 13) for later needle (or other medical implement) insertion in a confirmed proper location.

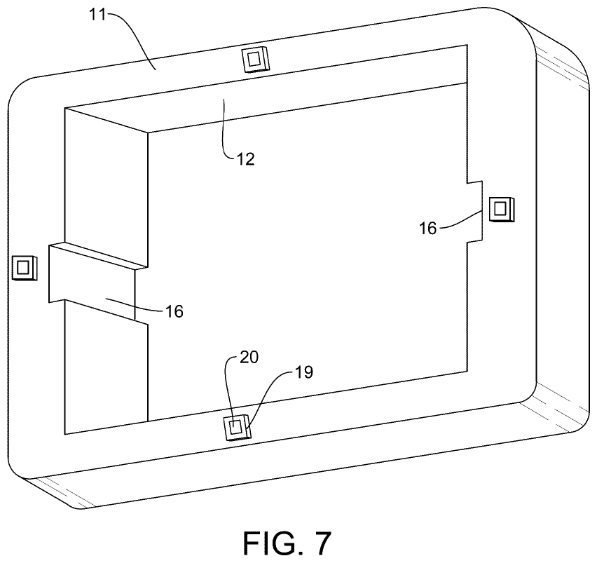

Turning now to FIG. 7, which shows an alternative embodiment of the outer frame 11 without the marking tool present. In this embodiment, the frame 11 retains the inner perimeter 12 and slots 16 sized to receive both an ultrasound probe 31 and marking tool 13, but the frame 11 also has four (4) square-shaped protrusions 19 on its front face. Each of the protrusions 19 have a square-shaped central opening 20 that may serve as a skin marker similar to the circular opening 18 on the marking tool 13. During the use of the ultrasound probe 31, when the frame 11 shown in this embodiment is used to mark the skin by any method, including, but not limited to, pressure, inking, spraying, and the like, the square openings 20 will indicate the center of the top, bottom, left, and right sides of the frame 11, respectively. Of course, the protrusions 19 and central openings 20 may be any shape without straying from the scope of the present disclosure. Additionally, the frame 11 may be wider on the left and right sides to accommodate both the slots 16 and the left and right protrusions 19.

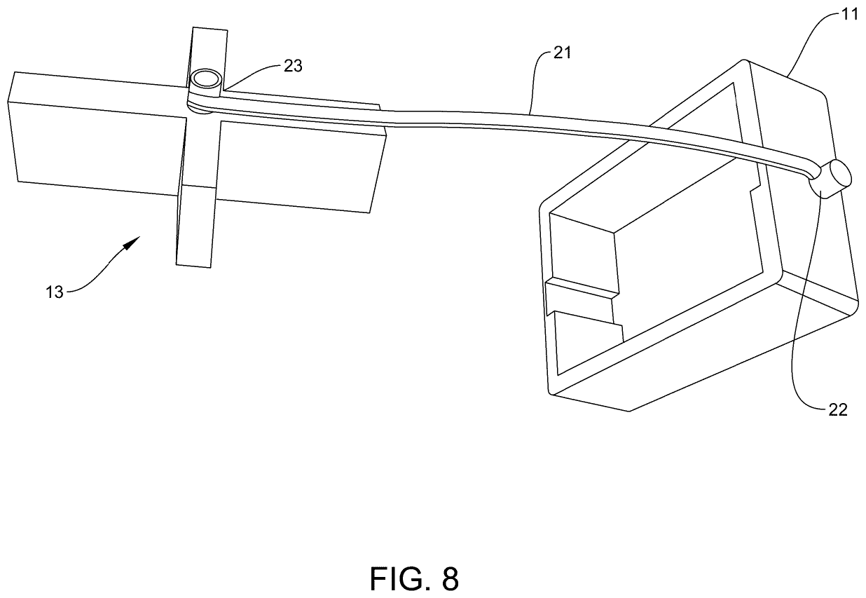

FIG. 8 shows another embodiment of the present disclosure with the frame 11 and marking tool 13 separated; however, in this embodiment, the frame 11 is connected to the marking tool 13 by a strap 21. The strap 21 is connected to both a protrusion 22 on a side of the frame 11 and a protrusion 23 on the rear of the marking tool 13. The strap 21 may be made of any material without straying from the scope of the present disclosure, including, but not limited to, flexible nylon, plastic, and the like. In some embodiments, the strap 21 may be connected to the frame 11 without the protrusion 22 and/or the marking tool 13 without the protrusion 23. For example, the strap 21 may be connected to the frame 11 and the marking tool 13 by any method without straying from the scope of the present disclosure, including, but not limited to, clips, adhesives, integrated slots, soldering, and the like. Additionally, in some embodiments that utilize the strap 21, the protrusion 23 on the rear of the marking tool 13 may or may not serve as a marker, similar to the protrusion 17 shown in FIGS. 1 and 2. In other embodiments, the protrusion 23 may be eliminated from the rear of the marking tool 13, as the attachment of the strap 21 to the frame 11 prevents the marking tool 13 from being inserted in the rear direction where the strap 21 is attached to it. In other words, the strap 21 may render the protrusion 23 unnecessary, especially in embodiments where the protrusion 23 is intended to serve as a marker. Regardless of the embodiment, having both the marking tool 13 and the frame 11 joined together by a strap 21 ensures that the marking tool 13 is easily accessible once the ultrasound probe is removed from the frame 11.

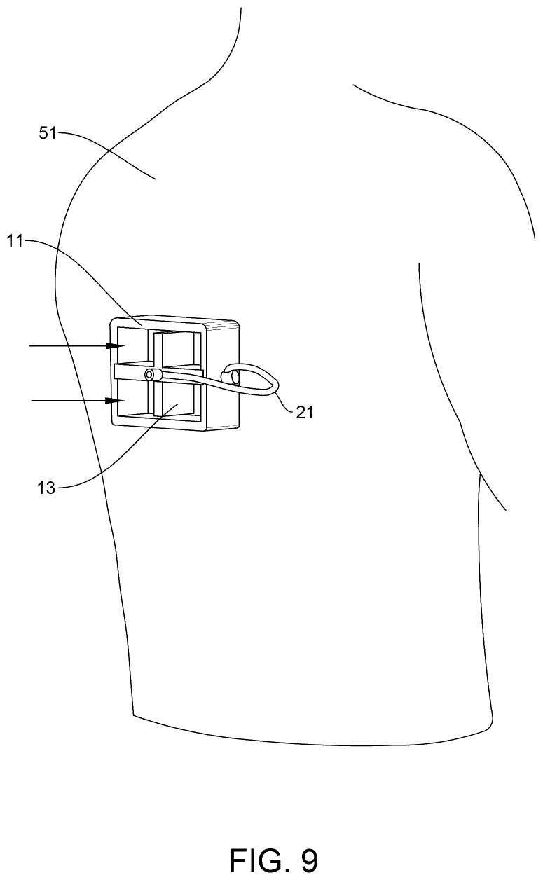

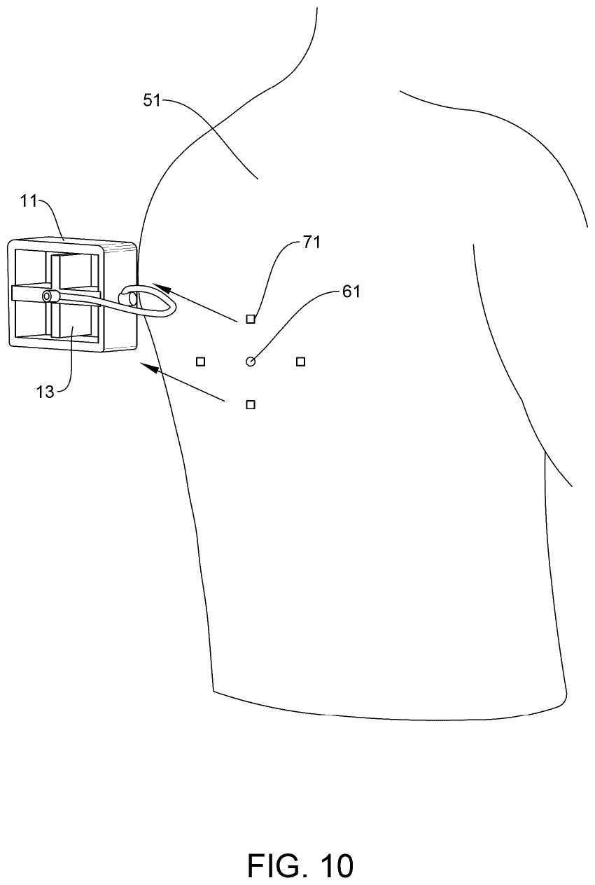

For example, as shown in FIGS. 9 and 10, once the ultrasound probe is removed, the marking tool 13 can be easily reached and inserted into the frame 11 in the direction of the arrows shown in FIG. 9 because both pieces are connected by the strap 21. In this embodiment, both the frame 11 and marking tool 13 comprise protrusion markers on their front faces (not shown). As previously discussed, when the marking tool 13 is pressed into the skin of the patient 51, an indentation 61 is made; however, in this embodiment, when the frame 11 is pressed into the skin of the patient 51, four (4) marks or indentations 71 are also made. Then, once both the frame 11 and marking tool 13 are removed in the direction of the arrows shown in FIG. 10, an individual can then compare the location of the indent 61 made by the marking tool 13 with the indents 71 made by the frame to see if the former is adequately centered. If the indent 61 is not adequately centered when compared with the indents 71, this may alert the individual that movement of the frame or displacement from its original position may have occurred either: (a) during the process of removing the ultrasound probe, or (b) sometime after making the outer indentations 71 with the frame 11 on the skin of the patient 51, but before making the central indentation 61, thus suggesting that rescanning and reverification of the markings is necessary to ensure the correct location for subsequent needle (or other medical implement) insertion.

It should be noted that while certain examples of the present disclosure have been presented using the present skin marking device with respect to epidural placement, that this device may be used for any number of applications, without straying from the scope of this invention. While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A skin-marking assembly comprising:
an outer frame;
a marking tool comprising a protrusion on a front side;
wherein the marking tool comprises a plurality of arms; and
the outer frame comprises two slots recessed from an inner perimeter of the outer frame, the two slots being sized and shaped to receive the plurality of arms.

2. The skin-marking assembly of claim 1 wherein the outer frame is sized to fit closely around an outer perimeter of an ultrasound probe.

3. The skin-marking assembly of claim 1 wherein the marking tool comprises a plurality of edges, each being sized to slidably fit within the inner perimeter of the outer frame.

4. The skin-marking assembly of claim 1 wherein the protrusion is sized and configured to form an indent in a skin of a patient.

5. The skin-marking assembly of claim 4 wherein the protrusion on the marking tool comprises a circular central opening.

6. The skin-marking assembly of claim 1 wherein the marking tool has a cross shape with a horizontal set of arms defining a left edge and a right edge, and a vertical set of arms defining a top edge and a bottom edge.

7. The skin-marking assembly of claim 6 wherein the two slots are sized and shaped such that either the horizontal set of arms or the vertical set of arms fit into the two slots.

8. The skin-marking assembly of claim 6 wherein the vertical set of arms is shorter than the horizontal set of arms.

9. The skin-marking assembly of claim 1 wherein at least one of the two slots is either Y-shaped, triangular, or straight.

10. The skin-marking assembly of claim 1 wherein the outer frame comprises a plurality of protrusions on a front face.

11. The skin-marking assembly of claim 10 wherein each of the plurality of protrusions are sized and configured to form an indent in a skin of a patient.

12. The skin-marking assembly of claim 10 wherein each of the plurality of protrusions on the outer frame comprise a square-shaped central opening.

13. The skin-marking assembly of claim 1 wherein the outer frame and the marking tool are connected by a strap.

14. The skin-marking assembly of claim 13 wherein the strap is connected to a protrusion on a side of the outer frame.

15. The skin-marking assembly of claim 13 wherein the strap is connected to a rear side of the marking tool.

16. A method of marking an area of a patient for placement of a spinal or epidural needle, comprising the steps of:
placing an outer frame of a skin marking assembly over an ultrasound probe;
identifying a spacing between vertebral structures of the patient using the ultrasound probe;
holding the outer frame in place on the patient while removing the ultrasound probe from the outer frame;
placing a marking tool into the outer frame; and
using the marking tool to mark the skin of the patient where the epidural is to be placed.

17. The method of claim 16 wherein the step of using the marking tool comprises pressing a protrusion of the marking tool into the skin of the patient to leave an indent in the skin.

18. The method of claim 16 further comprising using the outer frame of the skin marking assembly to mark the skin of the patient.

19. The method of claim 18 wherein the step of using the outer frame comprises pressing four protrusions of the outer frame into the skin of the patient to leave four indents in the skin.

20. The method of claim 19 further comprising comparing an indent made by the marking tool with the four indents from the outer frame to determine whether or not to repeat the step of using the ultrasound probe.
